(19) United States
(12) Patent Application Publication
(10) Pub. No.: US 2026/0021583 A1
(43) Pub. Date: Jan. 22, 2026

(54) SYSTEM AND METHODS FOR ENFORCING SAFETY IN INTELLIGENT SURGICAL ROBOTS

(71) Applicant: INTUITIVE SURGICAL OPERATIONS, INC., Sunnyvale, CA (US)

(72) Inventors: Omid Mohareri, San Francisco, CA (US); Muhammad Abdullah Jamal, Sunnyvale, CA (US)

(21) Appl. No.: 19/266,814

(22) Filed: Jul. 11, 2025

Related U.S. Application Data
(60) Provisional application No. 63/671,983, filed on Jul. 16, 2024.

(51) Int. Cl. B25J 9/16; A61B 34/35

(52) U.S. Cl. CPC B25J 9/1674; A61B 34/35; B25J 9/161; B25J 9/163

(57) ABSTRACT

Systems and methods are described for selecting tasks for using artificial intelligence. The system may include one or more repositionable structures configured to support respective instruments, and a control system operably coupled to the repositionable structure, the control system configured to receive a plurality of data streams; analyze, using a task generation machine learning model, the data streams to identify one or more tasks that may be performed by the one or more repositionable structures and generate respective risk values for the tasks, wherein a task generation constitution including a plurality of rules is input into the task generation machine learning model to control how the task generation machine learning model analyzes the data steams to identify the tasks; select, using a task selection machine learning model, an automated task based on the respective risk values; and control the one or more repositionable structures to perform the selected automated task.

### CROSS-REFERENCE TO RELATED APPLICATIONS

This application claims priority to and the benefit of the filing date of provisional U.S. Patent Application No. 63/671,983 entitled “SYSTEM AND METHODS FOR ENFORCING SAFETY IN INTELLIGENT SURGICAL ROBOTS,” filed on Jul. 16, 2024. The entire contents of the provisional application are hereby expressly incorporated herein by reference.

### TECHNICAL FIELD

The present disclosure relates generally to computer-assisted systems and more particularly to training and utilizing artificial intelligence to perform risk assessment of tasks associated with medical procedures and select tasks based on the risk assessment.

### BACKGROUND

Medical procedures often require various tasks to be performed by individuals or devices. Computer-assisted manipulator systems (“manipulator systems”), sometimes referred to as robotically assisted systems or robotic systems, may include one or more medical devices, equipment, and sensors that can be operated with the assistance of an electronic controller (e.g., computer or control system) to move and control functions of one or more instruments or medical devices. A computer-assisted medical system generally includes a robotic system with mechanical links connected by joints. An instrument is removably (or permanently) coupled to one of the links, typically a distal link of the plural links. In some embodiments, the computer-assisted medical systems are used in conjunction with one or more auxiliary devices (e.g., a surgical bed, an insufflator, etc.).

Conventional systems for monitoring medical environments do not provide any functionality relating to automatically determining which tasks need to be performed during a medical procedure, performing a risk assessment of the potential tasks that can be performed, and generating control outputs relating to specific tasks to be performed. For example, current medical environment task assistance methods do not take in, and cannot efficiently process, large sets of different data modalities, to determine task assignments and risk assessment of tasks for a procedure. Additionally, current systems do not consider the quality of the data the systems in determining which tasks should be performed.

Accordingly, there is a need for improved techniques that enable semi-autonomous, and fully autonomous generation, risk assessment, and selection of tasks while performing medical procedures. Such techniques can allow improved surgical outcomes, improved task workflows, and increased overall efficiency of performing medical procedures.

### SUMMARY

The following presents a simplified summary of various examples described herein and is not intended to identify key or critical elements or to delineate the scope of the claims.

In some aspects, the techniques described herein relate to a computer-assisted system for determining risk-based task classification and performing tasks, the system comprising (a) one or more repositionable structures configured to support respective instruments; and (b) a control system operably coupled to the repositionable structure, wherein the control system is configured to (1) receive a plurality of data streams from one or more data sources; (2) analyze, using a task generation machine learning model, the data streams to generate (i) one or more tasks that may be performed by the one or more repositionable structures and (ii) respective risk values associated with the one or more tasks, wherein a task generation constitution including a plurality of rules is input into the task generation machine learning model to control how the task generation machine learning model analyzes the data streams to generate the one or more tasks; (3) select, using a task selection machine learning model, an automated task based on the respective risk values; and (4) control the one or more repositionable structures to perform the selected automated task.

In some aspects, the techniques described herein relate to a method for determining risk-based task classification and performing tasks via a computer-assisted system comprising one or more repositionable structures configured to support respective instruments, and a control system operatively coupled to the one or more repositionable structures, the method comprising (1) receiving a plurality of data streams from one or more data sources; (2) analyzing, using a task generation machine learning model, the data streams to identify one or more tasks that may be performed by the one or more repositionable structures and determine respective risk values associated with the one or more tasks, wherein a task generation constitution includes a plurality of rules is input into the task generation machine learning model to control how the task generation machine learning model analyzes the data streams; (3) selecting, using a task selection machine learning model, an automated task based on the respective risk values; and (4) controlling the one or more repositionable structures to perform the selected automated task.

In some aspects, the techniques described herein relate to computer-readable media storing instructions that, when executed by a control system of a computer-assisted system, causes the control system to perform any of the methods described herein.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1 is a diagram of a computer-assisted system in accordance with one or more embodiments.

FIG. 2 is a schematic diagram of a system for generating, selecting, and performing tasks in furtherance of a medical procedure.

FIG. 3 is a schematic diagram of a robotic action module for generating actions tokens to the computer-assisted system.

FIG. 4 is a schematic diagram of an example task generation module for generating tasks for controlling a repositionable structure and associated risk values.

FIG. 5 is a schematic diagram of an example task selection module for selecting tasks to be performed by the computer-assisted system.

FIG. 6 is a schematic diagram of an example task assignment module for assigning tasks to actors.

FIG. 7A is a schematic diagram illustrating a process for detokenizing action tokens.

FIGS. 7B and 7C depict de-tokenized commands for controlling a repositionable structure.

FIG. 8 is a flow diagram of a method for risk-based task selection when performing a medical procedure.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, the terminology in this description is not intended to limit the invention. For example, spatially relative terms-such as “beneath”, “below”, “lower”, “above”, “upper”, “proximal”, “distal”, and the like—may be used to describe the relation of one element or feature to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. A device may be otherwise oriented and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. Additionally, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, implementation, system, or module may, whenever practical, be included in other embodiments, implementations, systems, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted systems and elements in terms of their state in three-dimensional space. As used herein, the term “position” refers to the location of an element or a portion of an element (e.g., three degrees of translational freedom in a three-dimensional space, such as along Cartesian x-, y-, and z-coordinates). As used herein, the term “orientation” refers to the rotational placement of an element or a portion of an element (e.g., three degrees of rotational freedom in three-dimensional space, such as about roll, pitch, and yaw axes, represented in angle-axis, rotation matrix, quaternion representation, and/or the like). As used herein, and for a device with a kinematic series, such as with a repositionable structure with a plurality of links coupled by one or more joints, the term “proximal” refers to a direction toward a base of the kinematic series, and “distal” refers to a direction away from the base along the kinematic series.

As used herein, the term “pose” refers to the multi-degree of freedom (DOF) spatial position and orientation of a coordinate system of interest attached to a rigid body. In general, a pose includes a pose variable for each of the DOFs in the pose. For example, a full 6-DOF pose for a rigid body in three-dimensional space would include 6 pose variables corresponding to the 3 positional DOFs (e.g., x, y, and z) and the 3 orientational DOFs (e.g., roll, pitch, and yaw). A 3-DOF position only pose would include only pose variables for the 3 positional DOFs. Similarly, a 3-DOF orientation only pose would include only pose variables for the 3 rotational DOFs. Further, a velocity of the pose captures the change in pose over time (e.g., a first derivative of the pose). For a full 6-DOF pose of a rigid body in three-dimensional space, the velocity would include 3 translational velocities and 3 rotational velocities. Poses with other numbers of DOFs would have a corresponding number of velocities translational and/or rotational velocities.

This disclosure occasionally refers to the disclosed techniques being applied to “patients” undergoing a “medical procedure.” It should be appreciated that these references are not intended to limit the application of the disclosed techniques to applied medicine contexts. For example, the described techniques can be applied to facilitate physician training, equipment testing and/or calibration, and/or other contexts. Accordingly, any reference to the term “patient” is done for ease of explanation and also envisions the application of the described techniques to a generic “subject.”

The word “task” is used herein to refer to a discrete portion of procedure that may be autonomously, semi-autonomously, or manually implemented in furtherance of a procedure. For example, a task may be to move an endoscope to a particular portion, to advance an instrument to a particular depth, to replace an instrument coupled to a manipulator, and so on. In some embodiments, a task is associated with component tasks to accomplish an overall goal. For example, a task to analyze a worksite may include component tasks related to moving an endoscope to view the worksite, advancing an instrument to predetermined depth, and enabling a functionality supported by the instrument.

Aspects of this disclosure are described in reference to computer-assisted systems, which can include devices that are teleoperated, externally manipulated, autonomous, semiautonomous, and/or the like. Further, aspects of this disclosure are described in terms of an implementation using a teleoperated surgical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including teleoperated and non-teleoperated, and medical and non-medical embodiments and implementations. Implementations on da Vinci® Surgical Systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperated systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

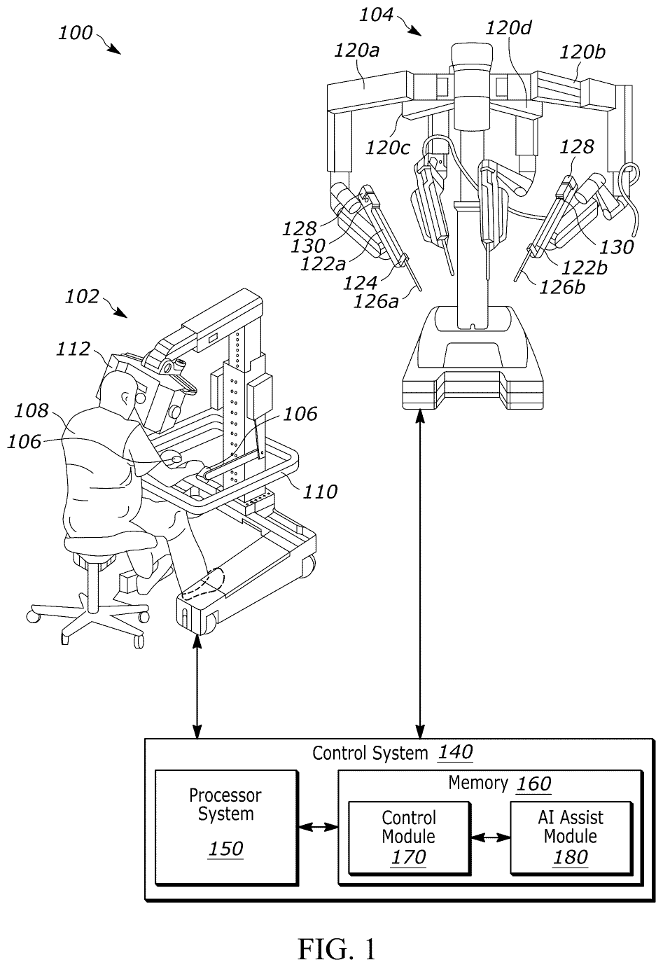

FIG. 1 is a simplified diagram of an example computer-assisted system 100, according to various embodiments. The computer-assisted system 100 may be a computer-assisted medical system for assisting with performing tasks in furtherance of medical procedures. Further, the computer-assisted system 100 may generate tasks that may be potentially performed in furtherance of the medical procedure and corresponding risk values associated therewith. In some examples, the computer-assisted system 100 is a teleoperated system. In medical examples, the computer-assisted system 100 can be a teleoperated medical system such as a surgical system. As shown, the computer-assisted system 100 includes a follower device 104 that can be teleoperated by being controlled by one or more leader devices (also called “leader input devices” when designed to accept external input), described in greater detail below. Systems that include a leader device and a follower device are referred to as leader-follower systems, and also sometimes referred to as master-slave systems. Also shown in FIG. 1 is an input system that includes a workstation 102 (e.g., a console), and in various embodiments the input system can be in any appropriate form and may or may not include the workstation 102.

In the example of FIG. 1, the workstation 102 includes one or more leader input devices 106 that are designed to be contacted and manipulated by an operator 108. For example, the workstation 102 may comprise one or more leader input devices 106 for use by the hands, the head, or some other body part(s) of operator 108. The leader input devices 106 in this example are supported by the workstation 102 and can be mechanically grounded. In some embodiments, an ergonomic support 110 (e.g., forearm rest) can be provided on which the operator 108 can rest his or her forearms. In some examples, the operator 108 can perform tasks at a worksite within a workspace near the follower device 104 during a procedure, by commanding the follower device 104 using the leader input devices 106. In a medical example, the worksite may be a surgical worksite associated with a patient.

A display device 112 is also included in the workstation 102. The display device 112 may be configured to display images for viewing by the operator 108. The display device 112 can be moved in various DOFs to accommodate the viewing position of the operator 108 and/or to provide control functions. In embodiments where the display device 112 provides control functions, the leader input devices 106 may include the display device 112. In the example of the computer-assisted system 100, displayed images may depict a worksite at which the operator 108 is performing various tasks by manipulating the leader input devices 106 and/or the display device 112. In some examples, images displayed by display device 112 may be received by the workstation 102 from one or more imaging devices arranged at a worksite. In other examples, the images displayed by the display device 112 may be generated by the display device 112 (or by a different connected device or system), such as for virtual representations of tools, the worksite, or for user interface components. In some embodiments the display device 112 may display one or more tasks for the operator 108 to perform with respect to any component of the computer-assisted system 100. The display device 112 may display one or more options for a user to provide an input of a user selection such as a preference for a task to be performed manually by the user or another personnel, or for a preference for a task to be performed automatically by or semi-automatically using one or more repositionable structures described further herein. Further, as described below, the display device 112 may display one or more tasks to be performed and associated task risk assessment values and allow for a user to input risk threshold values to compare the risk assessment values to the risk threshold values.

As illustrated, the computer-assisted system 100 also includes a follower device 104 that can be commanded by the workstation 102. In a medical example, the follower device 104 can be located near an operating table (e.g., a table, bed, or other support) on which a patient can be positioned. In some medical examples, the workspace is provided on an operating table, e.g., on or in a patient, simulated patient, or model, training dummy, etc. (not shown). As illustrated, the follower device 104 may include a plurality of repositionable structures 120 (sometimes referred to as “manipulator arms” in robotic embodiments). In some embodiments, the repositionable structures 120 may include a plurality of links that are rigid members and joints that can be individually actuated as part of a kinematic series. Additionally, each of the repositionable structures 120 is configured to couple to an instrument 122. While FIG. 1 illustrates a follower device 104 that has four repositionable structures 120a-120d, in other embodiments, the follower device 104 may include one, two, three, four, five, six, or additional or fewer repositionable structures 120a-120d.

The instrument 122 can include, for example, a working portion 126 and one or more structures for supporting and/or driving the working portion 126. Example working portions 126 include end effectors that physically contact or manipulate material, energy application elements that apply electrical, RF, ultrasonic, or other types of energy, sensors that detect characteristics of the workspace environment (such as temperature sensors, imaging devices, etc.), and the like. In various embodiments, examples of instruments 122 include, without limitation, a sealing instrument, a cutting instrument, a sealing-and-cutting instrument, an energy instrument for applying energy, a gripping instrument (e.g., clamps, jaws), a stapler, an imaging instrument such as one using optical, RF, or ultrasonic imaging modalities, a sensing instrument, an irrigation instrument, a suction instrument, and/or the like. In addition, the instrument 122 may include a transmission mechanism 128 that can be coupled to a drive assembly 130 of the respective repositionable structure 120a-120d. The drive assembly 130 may include a drive and/or other mechanisms controllable from workstation 102 that transmit forces to the transmission mechanism 128 to articular or otherwise actuate the instrument 122.

As illustrated, each instrument 122 may be mounted to a portion of a respective repositionable structure 120a-120d. In FIG. 1, this is shown with the drive assembly 130 physically coupled to the transmission mechanism 128. The distal portion of each repositionable structure 120a-120d further includes a cannula mount 124 to which a cannula (not shown) is mounted. When a cannula is mounted to the cannula mount 124, a shaft of the instrument 122 passes through the cannula and into a workspace.

In various embodiments, one or more of the working portions 126 of the instruments 122 may include an imaging device for capturing images. The imaging device may include any sensing technology capable of acquiring an image. Example imaging instruments include an optical endoscope, a hyperspectral camera, an ultrasonic sensor, etc. Imaging instruments may comprise monoscopic imagers, stereoscopic imagers, and/or the like. Imaging devices based on radiofrequency domains may capture images in any frequency spectrum, including visible light, infrared light, ultraviolet light, and/or the like. The imaging device may include an illumination source to light the region being imaged. In embodiments where the working portions 126 of one or more of the instruments 122 include an imaging device, the instrument 122 may be configured to capture images of a portion of the workspace for display via the display device 112.

In some embodiments, the repositionable structures 120a-120d and/or instruments 122 can be controlled to move the working portion 126 in response to manipulation of the leader input devices 106 by the operator 108 which may be used to perform semi-automatic tasks with input from the operator. Accordingly, the repositionable structures 120a-120d and/or instruments 122 may be said to “follow” the leader input devices 106 through teleoperation. This enables the operator 108 to perform tasks at the worksite using the repositionable structures 120a-120d and/or instruments 122. For a surgical example, the operator 108 can direct the repositionable structures 120a-120d of the follower device 104 to move the working portions 126 as part of a surgical procedure performed at an internal surgical site that is entered via one or more minimally invasive apertures or natural orifices. It should be appreciated that, in some embodiments, the follower device 104 may include non-teleoperated components that the operator 108 or other medical professional must manually manipulate to a desired pose.

In some embodiments, a repositionable structure 120a of the computer-assisted system 100 may be configured to support a working portion 126a that includes an imaging device (also referred to herein as an “imaging device 126a”). For convenience, an instrument 122 that includes an imaging device is also referred to as an “imaging instrument” herein. The control system 140 may be configured to command the repositionable structure 120a and/or the imaging instrument 122 comprising the imaging device 126a to automatically position and/or orient (“pose”) the field of view (FOV) of the imaging device 126a to provide images of the workspace and/or other instruments 122.

In the illustrated embodiment, a control system 140 is communicatively coupled to the workstation 102. In other embodiments, the control system 140 may be provided as a component of the workstation 102 and/or the follower device 104. During teleoperation, as the operator 108 moves the leader input device(s) 106, one or more sensors configured to detect the leader input device(s) 106 generate spatial and/or orientation movement data that is provided to control system 140. The control system 140 may interpret the spatial and/or orientation information to determine and/or provide control signals to the follower device 104 to control the movement of repositionable structures 120a-120d, instruments 122, and/or working portions 126. In addition to the components of the follower device 104, in some embodiments, the control system 140 is configured to interpret inputs received from the workstation 102 to control operation of one or more auxiliary devices (not depicted) utilized in a procedure. For example, the workstation 102 may be used to control a pose of a surgical bed or operation of an insufflator. The workstation 102 may be used to provide user input for performing one or more manual or semi-automated tasks that require input from users or personnel.

In one embodiment, the control system 140 supports one or more wired communication protocols, (e.g., Ethernet, USB, and/or the like) and/or one or more wireless communication protocols (e.g., Bluetooth, IrDA, HomeRF, IEEE 1102.11, DECT, Wireless Telemetry, and/or the like) for communications between the control system 140 and the workstation 102 and/or the follower device 104.

In some embodiments, the control system 140 may be implemented at one or more computing systems. For example, one or more computing systems may be used to control the follower device 104. As another example, one or more computing systems may be used to control components of the workstation 102, such as movement of a display device 112. Collectively, these component computing systems may comprise the control system 140.

As illustrated, the control system 140 includes a processor system 150, a memory 160, and an artificial intelligent (AI) assist module 180. The memory 160 may store a control module 170. The processor system 150 may include one or more processors having different processing architectures for processing instructions. For example, the one or more processors may be one or more cores or micro-cores of a multi-core processor, a central processing unit (CPU), a microprocessor, a field-programmable gate array (FPGA), an application-specific integrated circuit (ASIC), a digital signal processor (DSP), a graphics processing unit (GPU), a tensor processing unit (TPU), and/or the like.

In some embodiments, the processor system 150 includes circuitry to support one or more communication interfaces (e.g., Bluetooth interface, infrared interface, network interface, optical interface, etc.). Additionally, a communication interface of control system 140 may include an integrated circuit for connecting the control system 140 to a network (not shown) (e.g., a local area network (LAN), a wide area network (WAN) such as the Internet, mobile network, or any other type of network) and/or to another device, such as the workstation 102 and/or the follower device 104.

Additionally, the memory 160 may include non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, a floppy disk, a flexible disk, a magnetic tape, any other magnetic medium, any other optical medium, programmable read-only memory (PROM), an erasable programmable read-only memory (EPROM), a FLASH-EPROM, and/or any other memory chip or cartridge. The non-persistent storage and persistent storage are examples of non-transitory, tangible machine-readable media that can store executable code that, when run by one or more processors (e.g., processor system 150), can cause the one or more processors to perform one or more of the techniques and/or methods disclosed herein.

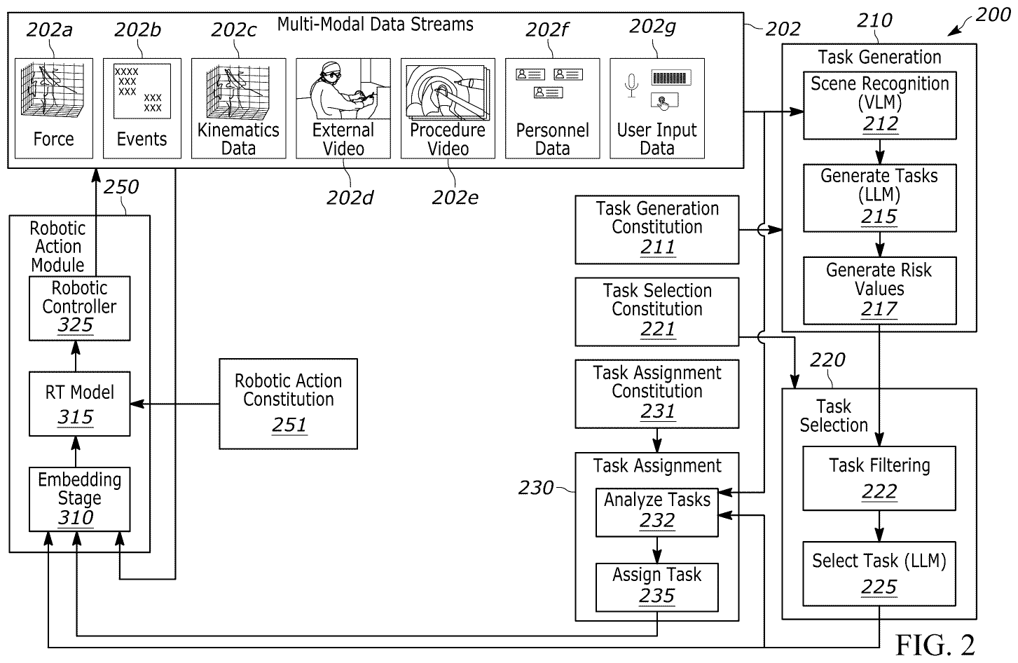

The AI assist module 180 may implement one or more machine learning models and/or training routines therefor. For example, the AI assist module 180 may implement one or more neural networks, deep learning models, decision trees, support vector machines, linear regression, generative AI models, reinforced learning models, random forests, Naïve Bayes models, large language models (LLMs), generative adversarial networks, foundation models, image recognition models, linear discriminant analysis models, creative applications, autoregressive models, supervised or unsupervised learning models, multimodal models, vision language models (VLMs), vision foundation models (VFMs), large multi-modal models (LMMs), Transformer models (including Robotic Transformer models), or another machine learning or AI model for performing the methods described herein. The structure of the one or more machine learning is described in more detail with respect to FIGS. 2-7C. The AI assist module 180 may include dedicated processors and memory for storing and performing AI processes, or the AI assist module 180 may utilize resources of the processor system 150 and the memory 160 to store and/or perform any processing or tasks required to perform the methods described herein. In some embodiments, the training routines are executed by another computing system (e.g., a server system) and the trained machine learning models are loaded into the AI assist module 180 prior to performing the medical procedure.

Additionally, the control system 140 may also include one or more input devices (such as a touchscreen, keyboard, mouse, microphone, touchpad, electronic pen, or any other type of input device) and/or output devices (such as a display device, a speaker, external storage, a printer, or any other output device). In some embodiments, the control system 140 may be implemented on a particular node of a distributed computing system (e.g., a cloud computing system). As another example, different functionalities associated with the control system 140 may be implemented on different nodes of the distributed computing system. Further, one or more elements of the aforementioned control system 140 may be located at a remote location and connected to the other elements over a network.

In an endoscopic surgery example, the imaging instrument comprising the imaging device 126a may be inserted into the patient prior to the other instruments 122, including a second instrument 122b comprising a second working portion 126b. The second instrument 122b can include any appropriate working portion 126b, and can even include a second imaging device. Accordingly, the imaging device 126a may be maneuvered to positioned to identify a target to which other instruments may interact with as part of another task. The control system 140 may, for example, automatically command the corresponding repositionable structures 120a and 120b to position respective instruments 122a and 122b to perform one or more tasks in tandem, or sequentially based on the specific task, instruments, and positions of the repositionable structures 120a and 120b. In examples, the control system 140 may perform AI processes and algorithms via the AI assist module 180 to generate tasks that can be potentially performed during a medical procedure and corresponding risk values associated therewith. Additionally, the control system 140 may filter the generated tasks into categories indicative of whether the task can be performed automatically by follower device 104, semi-automatically with user assistance or input to the system 100, and tasks that are to be performed entirely manually by personnel. As will be described below, this categorization may be determined based on the risk values of the respective tasks. The AI assist module 180 may then select tasks to be performed in furtherance of the medical procedure and assign the task to a particular actor (e.g., a particular repositionable structure 120, a particular instrument 122, a particular person, etc.).

For tasks that are to be performed semi-automatically or manually, the control system 140 may further analyze the available personnel to assign the task to the most appropriate individual for performing the task. For example, the control system 140 may assign tasks to personnel that have suitable skills and/or training to perform the task. As another example, the control system 140 may assign tasks from among suitable personnel based on their availability (e.g., whether or not the person is performing another task).

The disclosed techniques enable the control system 140 to perform real-time operations (e.g., within 5 ms, within 10 ms, within 20 ms, etc.). Accordingly, the control system 140 is able to identify and assign tasks to appropriate personnel in response to dynamic conditions during the course of a medical procedure. Such techniques improve the efficiency of operating the computer-assisted system or instrument, simplify user control of the computer-assisted system 100, improve the efficiency of the medical procedure by streamlining task workflow, and may reduce the required personnel in a medical environment for performing a procedure. Further, although a surgical example is shown, the disclosed techniques provide an improvement to the computer-assisted system 100 in the non-surgical aspects of the procedure, and can be used to improve computer-assisted systems applied in non-medical contexts.

FIG. 2 is a schematic diagram of a system 200 for generating, selecting, and performing tasks in furtherance of a medical procedure. The tasks may be categorized as to be performed manually, semi-automatically with user input or guidance, or entirely automatically by a robotic structure or system (such as a repositionable structure 120 or instruments 122). The system 200 includes a plurality of modules that may be executed by the control system 140 (e.g., via the AI assist module 180 of FIG. 1) to generate, select, assign, and perform one or more tasks associated with a medical procedure based on the associated risk values. The system 200 includes sources of multi-modal data that form one or more multi-modal data streams 202, a task generation module 210, a task selection module 220, a task assignment module 270, and a robotic action module 250.

It should be appreciated that in other embodiments, additional or fewer modules may be implemented. Further, in other embodiments, one or more of the described modules may be combined into a single module.

The system 200 includes one or more sources of multi-modal data 202. For example, the multimodal data 202 may include a data stream 202a indicative of force exerted upon an instrument, a data stream 202b indicative of system events generated by the repositionable structure and/or a control system thereof, a data stream 202c indicative of kinematic data associated with the repositionable structure and/or instruments or auxiliary devices associated therewith, an external video data stream 202d, a procedure video data stream 202e (such as image data generated by an endoscope), a data stream 202f indicative of personnel and data associated with personnel that may be available for performing the procedure, a data stream 202g indicative of user input data such as via a user interface, and/or other sources of data that indicate a state of a procedure facilitated by the repositionable structures. Additionally, the data streams 202 may include data retrieved from one or more data based such as preoperative data (e.g., preoperative images, video, patient information, etc.). In examples, the data streams 202a-202g may include system data (e.g., data associated with system events, system capabilities, etc.), and data associated with a medical environment (e.g., via video data, image data, available instruments, etc.). In examples, the multimodal data 202 may include one or more data streams that provide layouts of medical environments or rooms and/or information associated with one or more medical systems and instruments that may be available for a procedure.

The system 200 may be configured to route the multimodal data 202 to a task generation module 210 configured to output one or more potential tasks that may be performed in furtherance of the procedure, and associated risk values for the tasks, based on a procedure state represented by the input multimodal data 202. The task generation module 210 may identify tasks that may be performed in the near term (e.g., tasks that respond to conditions detected in an input set of multi-modal data) and/or tasks that may be performed in the long term (e.g., tasks that will need to be performed later in the procedure after one or more near-term tasks are performed). The task generation module 210 may further generate tasks to be performed in the future for a medical procedure scheduled to be performed at a later time or date. By also generating long-term tasks, the system 200 is able to track preparedness for performing the long-term task and generate additional tasks to prepare the control system for performing the long-term task as the procedure advances closer to the appropriate time for performing the long-term tasks. In some embodiments, the task generation module 210 may include component models to facilitate the analysis. For example, the task generation module 210 may include a scene recognition model 212 to parse image data to generate natural language descriptions of the scene represented by the image data (and the corresponding locations in the image data) and a task generation model 215 configured to actually generate the one or more tasks. As one example, the scene recognition model 212 may detect and identify objects in a medical environment such as an operating room (OR) to generate an inventory of objects in the OR.

The task generation module 210 may analyze the multimodal data 202 to generate the potential tasks to be performed according and the corresponding risk values to a set of rules. For example, a task generation constitution 211 may be input into the task generation module 210 as part of a prompt that also includes at least a portion of the multimodal data streams 202.

In some embodiments, a data stream input into the task generation module 210 may include an embedding indicative of the medical procedure, or of one or more phases of the medical procedure. One such technique for detecting the medical procedure type is described in U.S. Provisional Application No. 63/663,996, the entire disclosure of which is hereby incorporated by reference. Accordingly, in these embodiments, the procedure information may be input into the task generation model 215 to generate tasks based on the particular medical procedure (and/or phase thereof) and according to the rules in the task generation constitution 211.

The generation module 210 may further include a risk value model 217 that receives the generated tasks from the task generation model 215 and determines risk values for each task. It should be appreciated that while FIG. 2 depicts the risk value model 217 as a separate model from the task generation model 215, in some embodiments, a single prompt to the LLM may both generate the potential tasks and the corresponding risk values based on the task generation constitution 211. The risk values may be indicative of the ability of the task to be performed in a safe and/or autonomous manner. For example, the risk value may be indicative of a predicted difficulty in autonomous operation (e.g., because the task requires a high degree of precision, one or more obstacles may complicate the performance of the task, etc.), and/or be indicative of a presence or quality of a data stream needed to perform a task (e.g., a required data is not available, a useful, but not required data stream is not available, a quality of the data within the data stream is not of sufficient quality (e.g., image data is unfocused or noisy, force data is outside an expected range, an object is occluding a target in the field of view in the image data, etc.)). Similarly, for manual and/or semi-autonomous tasks, the risk value may be based on the available personnel to perform a task, the training and skills of the personnel, personnel performance scores for earlier portions of the procedure and/or historical procedures, etc. Accordingly, task generation constitution 211 may define rules for how to assign risk values based on the various inputs to the task generation model and/or output from the scene recognition model 212.

The system 200 may then provide the output tasks (e.g., natural language descriptions of an action to be performed) and associated risk values to a task selection module 220 to select which tasks should actually be implemented by the control system and/or an operator thereof based on the risk values. As illustrated, the task selection module may include a task filter model 222 configured to filter the tasks generated by the task generation model 215 and a task selection model 225 configured to select one or more valid tasks to be performed by the control system and/or an operator thereof.

More particularly, the task selection module 220 may input a task selection constitution 221 input into a task selection model 225 along with one or more input tasks and the corresponding risk values to select which tasks are to be performed. The task selection constitution 221 may include one or more sets of rules that control how the task assignment module 220 filters and selects the identified tasks. In examples, the rules of the task selection constitution 221 may cause the task selection module 220 to filter the various tasks according to one or more available data streams, available personnel, user inputs and preferences, device models, available instruments etc. Additionally, the rules may include a set of rules indicative of how to assess the risk values when selecting between multiple potential tasks. As one example, a task that would normally be performed autonomously may instead be classified as being a semiautonomous task in response to the risk value being below a threshold value. Regardless, the task selection module 220 may then provide the selected tasks to the task assignment module 230, and/or the robotic action module 250.

The task assignment module 230 may be configured to analyze the one or more selected tasks to assign the tasks to respective personnel according to rules of a task assignment constitution 231 and based on a specific task and associated risk value. Additionally, one or more data streams of the plurality of data streams 202 may be input into the task assignment module 230 to assist in the task assignment analysis. The task assignment module 230 includes a task analysis model 232 configured to analyze to identify which personnel (including combinations of various personnel) are capable of implementing the selected tasks and a task assignment model 245 configured to assign the task to respective personnel. The task analysis model 232 may identify different requirements for each task (e.g., personnel skillsets and qualifications, instruments, instrument and system capabilities, etc.) and may utilize the risk values associated with the various tasks to identify which personnel should be considered for performing a given task.

In some embodiments, a particular task may require multiple personnel to perform different aspects of a given task. Accordingly, the task analysis model 232 may be configured to divide the task into subtasks that are assigned to different personnel to minimize risk based on one or more associated risk values. In examples, the risk value for a given task may be associated with specific personnel and the risk value may further be considered in determining what personnel the task assignment module 230 assigns a task to or if a task should be performed automatically or semi-automatically.

The task assignment module 230 further includes an assign tasks module 235 that analyzes the personnel requirements for the input tasks (and/or subtasks thereof), associated task risk values, and assigns the various tasks to appropriate personnel, repositioning structures, and/or instruments according to one or more rule sets of the task assignment constitution 231. That is, the task assignment module 230 may be configured to input both the selected tasks (and/or subtasks thereof) and the task assignment constitution 231 to the task assignment module 235 to determine the appropriate task assignments. For example, if it is determined that a risk value for a selected task is below a threshold, then the task assignment constitution 231 may include a higher qualification threshold for the task to be assigned to particular personnel.

It should be appreciated that if required instruments are not present or available, the rules of the task assignment constitution 231 may instruct the task assignment module 230 to send an indication to the task generation module 210 and/or the task selection module 220 to generate and/or select alternative tasks that can be performed based on the current instrument availability. It should be appreciated that if appropriate personnel are not present, or not available to perform a task, the task assignment module 235 may assign the task to a robotic structure or system (if possible), or may attempt to assign the task to other personnel based on a task risk value. As another example, if a task is associated with a high risk value due to poor quality image data, the task assignment constitution 231 may still instruct the task assignment model 235 to assign the task to the appropriate instrument for performing the task, but additionally cause the task generation module 210 to generate tasks to clean and/or otherwise address the image quality.

In examples, the outputs of task selection module 220 and/or the task assignment module 230 may be provided to the operator, such as via the workstation 102 of FIG. 1. The operator may then approve of the tasks to be performed and/or the task assignments prior to implementing the selected tasks. Additionally, the risk values associated with various tasks may be provided to a user and a user may approve or reject the various tasks based on the provided risk values and associated task assignments. If the operator disapproves the selected tasks, or specific task assignments, the task selection module 220 may provide alternative tasks that can be performed, and/or the task assignment module 230 may provide different assignments of the tasks and further provide the risk values associated with the alternative tasks and/or alternative task assignments. In some embodiments, if the operator disapproves the assignment of the tasks, the operator may be able to manually assign the task to personnel or a robotic system via the display device 112. In such examples, the task generation module 210 may generate new tasks and corresponding risk values for presentation to the user.

To perform these semi-autonomous and fully autonomous tasks, the system 200 includes the robotic action module 250. As illustrated, the robotic action module 250 may be configured to receive the selected tasks from the task selection module 220, task assignments from the task assignment module 230, and/or the multimodal data 202. The robotic action module 250 may input the embeddings of the various inputs and a robotic action constitution 251 into a RT model 315 to generate one or more action tokens. For example, the action tokens may be natural language descriptions of a robotic action to be performed by the manipulator system in furtherance of a selected task. The robotic action module 250 may embed the input data streams 202 via an embedding stage 310 for input into an RT model 315. The robotic action module 250 may then convert the action tokens to command one or more components of a computer-assisted robotic device (such as the follower device 104 of FIG. 1) to perform one or more of the selected tasks.

It should be appreciated that in some embodiments, one or more of the modules 210, 220, 230, may be combined into a single module, for example, by implementing chain of thought (CoT) or other recurrent prompting techniques such that a single prompt performs the described functionality corresponding to the individual modules. Additionally, in other embodiments, the system 200 may include additional modules that generate inputs to the robotic action module 250 to implement autonomous tasks. For example, the system 200 may include a data modality selection module that selects which data streams are embedded and input into the RT model 315. One such embodiment of a system that includes these additional modules is described in U.S. Provisional Application No. 63/663,539, the entire disclosure of which is hereby incorporated by reference.

Generally, the constitutions of the system 200 (e.g., the constitutions 211, 221, 231, 251) may include various sets of rules according to different types or categories of rules. For example, the constitutions may include foundational rules that define allowable robotic actions (e.g., limitations on instrument operation for patient and user safety, such as Asimov's three rules), safety rules that define safe operation of the computer-assisted medical system (e.g., spatial limitations, sanitization standards, etc.), embodiment rules that define capabilities and limitations of the computer-assisted medical system, associated instruments and devices (e.g., degrees of freedom of robotic arm movement, imaging capabilities of endoscopes and imaging devices, volume capabilities of extraction and injection devices, etc.), and/or user preference rules (e.g., rules defined and input by a user or personnel for performing a procedure or task).

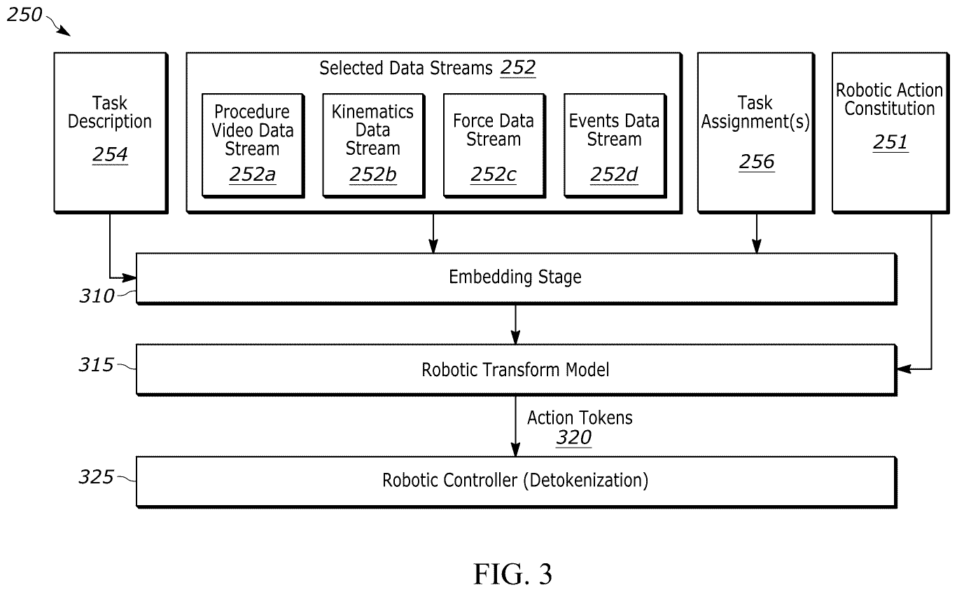

FIG. 3 is a schematic diagram of a robotic action module 250 (such as the robotic action module 255 of FIG. 2), for generating actions tokens to control a repositionable structure (such as the repositionable structures 120 of FIG. 1). The robotic action module 250 may be implemented as part of the AI assist module 180 of FIG. 1. As described with respect to FIG. 2, the robotic action module 250 may be configured to receive as inputs one or more data streams forming the multi-modal data 202, an indication of a selected task 254, and an indication of a one or more specific assigned tasks 256, and a constitution such as the robotic action constitution 251 of FIG. 2.

As illustrated, the robotic action module 250 includes an embedding stage 310 that embeds the various data modalities (e.g., text, image, audio, video, sensor data, etc.) into a common data space for input into an RT model 315. The embedding stage 310 may be configured to project the input data streams into a common vector space. In examples, the embedding stage 310 may implement one or more embedding methods including, without limitation, a word2vec, GloVe, ELMo, BERT, principal component analysis, singular value decomposition, a transformer model, Doc2Vec, paragraph vectors, convolutional neural networks, pre-trained models for image embedding, node embeddings, iknowledge graph embeddings (e.g., TransE, TrensR, DistMult, etc.), word embeddings, graph embeddings, entity embeddings, or another type of embedding supported by the RT model 315. It should be appreciated that while the embedding stage 310 is depicted as a single block, each data stream 252 may be associated with a respective embedding model for generating embeddings of the respective data type included within the embedding stage 310. Further, while embedding stage 310 is depicted as a component of the robotic action module 250, in some embodiments, the embeddings generated by the embedding stage 310 are input into the various models of the task generation module 210, the task selection module 220, and the task assignment module 230.

The various embedding models implemented by the embedding stage 310 may be trained and/or fine-tuned using historical data of prior procedures. As one example, an embedding model to analyze a procedure video data stream 252a may be trained to identify objects (e.g., instruments, ports, anatomical features, etc.) that are expected to be seen during the procedure. In this example, the embedding model for the procedure video data stream 252a may be trained and/or fine-tuned using historical image data in which the pixels representative of the corresponding objects are labels. The embedding model may then be trained or tuned in any suitable manner that minimizes loss with respect to the set of ground truth labels.

As another example, an embedding model for a kinematic data stream 252b, force data stream 252c, and/or an events data stream 252d may be trained using labeled segments from historical procedures performed using the same type of manipulator system associated with the system 200. For example, after a historical procedure has been performed, the set of kinematic, force, and/or event data captured during the historical procedure may be correlated, compiled, and aligned in time. For some types of conditions, the set of data may then be labeled by identifying time segments at which a particular condition occurred (e.g., an instrument or end effector being operated in a particular manner, performance of a particular phase of a procedure, transition to a new phase of a procedure, etc.). In some embodiments, the condition may relate to identifying a particular phase of a medical procedure. By building a library of labeled time segments, an embedding model can be trained to identify the conditions in the corresponding data streams when implemented in the robotic action module 250. In these embodiments, the embedding model may include a time-series analysis model (such as a Transformer model or a recurrent neural network (RNN)) configured to capture the temporal relationship between data within the labeled time segments.

For other conditions (such as alerts, errors, anomalies, abrupt and/or significant changes in operation, or other instantaneous conditions), the representation of that condition in the historical data may be labeled such that the embedding model can be trained to detect the labeled conditions. In either case, the training system may implement self-supervised learning to identifying the conditions and/or fine-tuning based on the procedure type and/or manipulator system such that the identification is tailored to the procedure/manipulator system associated with the system 200.

The robotic action module 250 then provides the embedded data output from the embedding stage 310 as an input to the RT model 315. The RT model 315 may implement any type of robotic transformer architecture configured to convert embeddings into action tokens for control of a robotic system. One such suitable architecture is the RT-2 architecture by DeepMind, but other RT model architectures are envisioned. The RT model 315 is configured to process the embedded data from the embedding stage 310 to generate action tokens 320 for controlling the instruments, auxiliary devices, and/or repositionable structures to perform the selected tasks. In some embodiments, the robotic action constitution 251 is also input into the RT model 315 and may include procedure rules, and embodiment rules, among other sets of rules that determine how the RT model 315 generates the action tokens 320 to perform certain tasks and procedures, given a specific manipulator system type. The action tokens 320 may be textual descriptions of robotic actions that the selected arm, instrument, and/or auxiliary device are to perform to implement the selected task. It should be appreciated that the particular tokens supported by the manipulator system may vary between models. Accordingly, the system 200 may be coupled to a library of RT models 315 each fine-tuned to generate action tokens supported by a different manipulator system type. As a result, the particular outputs of the tuned RT model 315 are adapted to the actual manipulator system associated with the system 200.

The robotic action module 250 then inputs the action tokens 320 to a robotic controller 325 that de-tokenizes the action tokens 320 into actual control commands the control operation of instruments, arms, and/or auxiliary devices according to the assigned tasks 256. In examples, to perform an assigned task 256, a de-tokenized action token may include a plurality of commands including movements of one or more repositionable structures, control of an action of an instrument (e.g., power up, power down, inject, extract, incise, etc.), control of an auxiliary structure (e.g., surgical bed, display device, etc.), or another command for performing a given task. In some embodiments, the de-tokenized control commands are signals suitable for various types of robotic control architectures that may be implemented at the manipulator system. For example, the de-tokenized control commands may be a signal input into a proportional-integral-derivative (PID) controller that controls a particular joint, or a model predictive control (MPC) signal, and/or other types of control signals. As described further herein, the de-tokenized commands may be specific to a given robotic system or machine. For example, a given system may include manipulators with more degrees of freedom of motion than another system, or an instrument, such as a camera, may have a wider field of view than another camera, and the de-tokenized commands may differ depending on the various capabilities and parameters for a given robotic system.

As illustrated, in some embodiments the robotic controller 325 may also accept the robotic action constitution 251 as an input to provide rules for how actions tokens are to be converted to specific parameter values. For example, the embodiment rules may include parameter range values for controlling function degrees of freedom associated with the controlled instruments. Accordingly, by dynamically adapting the embodiment rules, the system 200 is able to ensure that the action tokens can implemented across a wide variety of instrument types without needing to fine-tine the RT model 315 for each new instrument type.

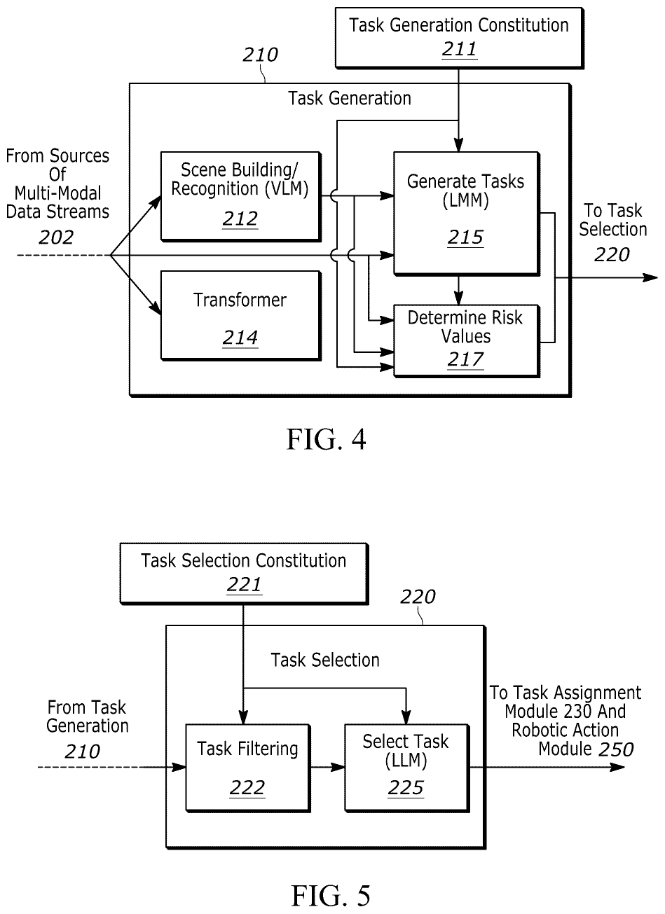

FIG. 4 is a schematic diagram of the task generation module 210 for determining tasks that can be performed based on a current state of the manipulator system and/or procedure as reflected by the multimodal data streams 202. The task generation module 210 additionally determines risk values associated with each task based on various factors as described herein. As shown in FIG. 2, the task generation module 210 is configured to receive the multi-modal data streams 202 and the task generation constitution 211 as inputs. Similar to the embedding stage 310 of the robotic action module 250, the task generation module 210 may embed the data streams 202 for input into a task generation model 215. In some embodiments, the embedding models used to embed the data streams 202 are the same as the embedding models included in the embedding stage 310 of the robotic action module 250. The task generation constitution 251 may provide rules for how the task generation module 210 embeds the data streams 202, for how the task generation model 215 generates tasks, and how the risk assessment model 217 determines risk values for the tasks.

For example, the embedding model for an endoscopic data stream may be routed to a vision-language model (VLM) 212 configured to output natural language descriptions of a scene depicted by the image data output by an endoscopic instrument. In surgical applications, for example, the VLM 212 may be configured to identify objects, such as surgical instruments and devices (e.g., surgical beds or tables, medical devices, display devices, etc.), individuals and personnel (e.g., clinicians, doctors, medical technicians, etc.) depicted by an image data stream. Additionally, the task generation module 210 may further include a VFM to perform various machine vision and scene recognition processes.

As another example, the embedding model of the event data stream may be configured to analyze the time series event data using a transformer model 214 to generate an output token identifying a particular phase of the procedure. It should be appreciated that while the term “transformer model” is used herein, in other embodiments, other model architectures suitable for analyzing temporal dependencies may be implemented. These output tokens may be input into the task generation model 215 to generate tasks that are in furtherance of operation typically associated with the identified phase. Similarly, the transformer model 214 may be configured to detect the completion of a phase or sub-phase of the procedure and generate an output token identifying the transition. These output tokens may be input into the task generation model 215 to begin generating tasks to implement actions associated with a subsequent phase or subphase. Additionally, the transformer model 214 may be configured to detect anomalous operation associated with phase of the procedure and output token indicative of the anomaly (e.g., a token indicative that anomalous operation is occurring, a token indicating a particular type of anomaly that is occurring, and so on). These output tokens may be input into the task generation model 215 to generate tasks to correct the anomaly.

In additional to generating tokens based on time-dependencies in the event data stream, the transformer model 214 may also be configured to output tokens based on an instantaneous representation in the event data stream. To this end, the event data in the event data stream may indicate the complete state of the robotic components of the manipulator system at any given time. Accordingly, the transformer model 214 may be configured to identify equipment operatively coupled to the control system (e.g., manipulator arms, instruments, auxiliary devices) to generate an inventory of devices. In some embodiments, the inventory may also be input to the task generation model 215 such that the task generation model 215 understands that capabilities of the control system and generates tasks that can be implemented thereby.

In addition to identifying equipment currently coupled to the control system, an embedding model for another data stream may be configured to detect equipment that is otherwise available for use in the procedure (e.g., sterilized equipment on-hand in an operating room, equipment available elsewhere at the site). For example, the on-hand equipment may be detected via an operating room video data stream or via an equipment scheduling data stream. Additionally, the task generation model 215 may receive data indicative of personnel that are currently available, will be available for a scheduled procedure, or are required to perform one or more tasks via the data stream 202f indicative of personnel and data associated with personnel.

The tokens generated by the embedding models may then be input into the task generation module 215. The task generation model 215 may be a LMM configured to directly accept image data streams or an LLM that accepts the natural language description(s) of the image data stream(s) generated by the VLM 212. The task generation model 215 may then generate tasks in furtherance of the detected (or subsequent) phase of the procedure based on the available equipment, personnel, and any other conditions indicated by the input data streams 202. The task generation constitution 211 may include sets of rules that control how the task generation model 215 generates the tasks from the detected phase of procedure, available equipment, personnel, and other conditions detected or indicated by the input data streams 202.

The task generation module 210 further includes a risk assessment model 217 that determines risk values associated with the various tasks generated by the task generation model 215. While FIG. 4 depicts the risk assessment model 217 as being separate from the task generation model 215, in some embodiments, the risk assessment associated with the risk assessment model 217 may be performed by including instructions related to risk assessment in the prompt input into the LMM (or other model) of the task generation model 215. The risk assessment model 217 may determine the risk values based on rules defined in the task generation constitution 211. The risk assessment model 217 receives, as input, the tasks from the task generation model 215, data streams from the multi-modal data streams 202, and embeddings from the VLM 212. The risk assessment model 217 determines the risk values for each task based on the one or more of the data streams (e.g., types of available data streams, quality of data in one or more data streams, etc.), the tasks from the task generation model 215 (e.g., complexity of a task, time required to perform a task, etc.), and from the scene building and object recognition (e.g., available personnel, available instrumentation, evaluation of obstacles in the environment, etc.).

The risk assessment model 217 may determine respective risk values as numerical values. For example, the risk assessment model 217 may assign each task a risk value between 1 and 10 with I being an indication of low risk, and 10 being an indication of high risk. In this case, the task generation constitution may include rules defining how to score the risk on the 1 to 10 scale. Additionally, the risk assessment model may assign each task more than one risk value depending on different sources of risk. For example, the risk assessment model 217 may assign a first component risk value associated with a quality of the data that will be relied upon to perform the task and a second component risk value associated with the capabilities of the actor (e.g., the repositionable structure 120, the instrument 122, or personnel) intended to perform the task. In these embodiments, the risk value may further include a composite risk value based on a combination of the component risk scores (e.g., by averaging). It should be appreciated that this risk value may be distinct from a confidence value output by many types of LMMs indicative of the confidence of the prediction model (e.g., a model that includes conformal prediction techniques) that the output information complies with the input prompt. That said, in some embodiments, this confidence value may be considered a component risk value.

While described as assigning numerical risk values to tasks, the risk assessment model 217 may assign other indicators of risk such as text classification (e.g., high risk, low risk, medium, risk, etc.) or another type of risk indicator for the tasks. The risk assessment model 217 may include an LLM that receives embeddings indicative of the various data streams, tasks, and the task generation constitution 211. The risk assessment model 217 may output the risk values as natural language text. By having fewer categories of risk levels, the task generation constitution 211 may utilize fewer input tokens, enabling more complex outputs of the task generation model 215 and/or risk assessment model 217.

The task generation constitution 211 may control how the task generation model 215 generates tasks and how the risk assessment model 217 determines the risk values and the format thereof. For example, the task generation constitution 211 may include foundational rules that define allowable robotic actions (e.g., limitations on instrument operation for patient and user safety, such as Asimov's three rules), safety rules that define safe operation of the computer-assisted medical system (e.g., spatial limitations, sanitization standards, etc.), embodiment rules that define capabilities and limitations of the computer-assisted medical system, associated instruments and devices (e.g., degrees of freedom of robotic arm movement, imaging capabilities of endoscopes and imaging devices, volume capabilities of extraction and injection devices, etc.), procedural rules (e.g., rules pertaining to the limitations and requirements for performing a specific procedural), and/or user preference rules (e.g., rules defined and input by a user or personnel for performing a procedure or task).

The foundational rules may include rules pertaining to the allowable robotic actions and limitations of robotic actions. For example, the foundational rules may define rules that prevent robotic actions from being performed that could harm a human or operator, rules that cause a robotic system to receive and follow orders provided by users, prevent a robotic system from harming a patient, prevent a robotic system from interfering with other devices and tasks, etc. The foundational rules may further include rules for calculating risk values based on the various foundational rules and limitations.

The safety rules may include rules that define actions and operations that are deemed safe for a given environment, operation, and scenario. The safety rules may additionally include rules that define actions and operations that are safe or unsafe based on available data streams, quality of data in data streams, and capabilities of available personnel and/or equipment. For example, the safety rules may include rules that restrict or define spatial limitations to movement or operation of a robotic system, rules that pertain to maintaining sterility of a robotic system or instruments, rules that define safe use of specific instruments and devices, rules that limit a robotic systems abilities pertaining to known limits of a specific robotic system model, etc. The safety rules may be specific to a scenario or environment such as a certain institution may have different safety standards than other medical facilities and therefore the safety rules may reflect the various standards of institutions, states, regions, governments etc. Additionally, the safety rules may also be specific to certain personnel present or performing tasks for an operation. For example, the safety rules may depend on the types of personnel and training of personnel available for performing an operation. The safety rules may further include how risk values are to be determined for performing various actions according to various safety standards and with the available robotic systems and personnel.

The embodiment rules may include rules that define capabilities and limitations pertaining to specific models of instruments, devices, and systems. For example, the embodiment rules may include rules pertaining to available degrees of freedom of motion, ranges of motion, imaging resolutions, video capabilities, audio recording parameters, etc. The embodiment rules may include rules that pertain to the dimensions and sizes of various instruments, devices, or system components to control how a robotic system or instrument moves in an environment, and to prevent components from contacting other objects as required. Additionally, the embodiment rules may include rules that specify and govern actions that may be performed by specific instruments, devices, and systems such as rules that control insection, suction, imaging, injection, extraction, application of radiation, heating, etc. In more specific examples, the embodiment rules may pertain to a field of view of a robotic system or instrument, one or more payloads of a robotic system or instrument, and/or a capability or limitation of one or more sensors. The embodiment rules may further include risk values and how to determine risk values for utilizing certain equipment or based on the abilities and limitations of specific embodiments for performing various tasks.

The procedural rules may include rules that define limitations and required actions for a given procedure. For example, the procedural rules may include rules that dictate certain actions or tasks must be performed, other actions must never be performed, and additional tasks or actions may optional be performed for performing a procedure. The procedural rules may further include rules that pertain to various tasks that may be performed autonomously, semi-autonomously, and/or manually.

The user preference rules may include rules pertaining to rules that are defined by a user or based on user input and preferences. For example, the user preference rules may include rules that prioritize use one instrument or device over another based on a user provided preference, that prioritize use of a specific robotic arm or a manipulator system, control how one or more models (such as the task selection model 220) filters tasks into automatic, semi-automatic, and manual tasks based on user input, how the task selection model 220 ranks tasks, how the task assignment model 230 assigns tasks, what is displayed to a user, how data and prompts are displayed to a user, etc.

The task generation model 215 may be a foundational machine learning model that is fine-tuned to generate the tasks and determine associated risk values for the tasks, based on the input data streams. For example, the task generation model 215 may implement conformal prediction techniques and/or Q-learning techniques to fine-tune the ability of the task generation model 215 to assign risk values to the generated tasks. Durning the tuning process, a safe task metric may be used as a validation parameter the tuning process evaluates to determine when the tuning is complete. The safe task metric may then be defined to be the percentage of tasks that are safe to be performed.

Accordingly, performing the Q-learning may include using a conservative Q-function that evaluates the system performance (e.g., based on the safe task metric) of generating tasks. The Q-learning approach provides an overall score for the system performance, and trains the system not to perform certain actions that have scores outside of the Q-function distribution. This helps prevent the system 200 from engaging in actions and tasks that the system 200 should not perform.

During the tuning process, sets of multimodal data 202 representative of typical scenario that arises during medical procedures may be input the task generation model 215 to generate a set of potential tasks that may be performed at different points of time. A second machine learning model may assess the generated tasks to determine whether the potential tasks can actually be safely performed (either in isolation or in view of the input multimodal data). Depending on the implementation, the tuning process may be complete when a tuning epoch satisfies a threshold safe task metric (e.g., 90%, 95%, 98%, etc.).

The conformal prediction training may include providing a measure of certainty for each decision, or multiple points along deciding, and filtering the tasks. The conformal prediction approach provides sets of probable outcomes for a given iteration for selection tasks, rather than a single prediction. This provides users insight into the uncertainty or confidence level associated with each prediction of system performance. The conformal prediction approach may be useful for providing the system a means by which it can evaluate and determine points during procedures, or tasks. For example, the uncertainty or confidence level output by the conformal prediction may be utilized as a risk value that is assigned to the task.

FIG. 5 is a schematic diagram of the task selection module 220 configured to select one or more tasks and/or sets of tasks generated by the task generation module 210 for implementation by the control system. As illustrated, the task selection module 220 may select the tasks in two stages. As shown in FIG. 2, the task selection module 220 is configured to receive the generated tasks and associated risk values from the task generation module 210 and the task selection constitution 221 as an input.

In the first stage, a task filtering model 222 is implemented to categorize the generated tasks for filtering. For example, the task filtering model 222 may categorize each task is capable of being performed autonomously, semi-autonomously, manually, or not at all. In some embodiments, the task selection constitution 221 includes rules that define how to classify different categories of tasks as being capable of autonomous, semi-autonomous, or manual performance. Tasks that are not capable of being performed may be filtered out. It should be appreciated that while the task generation model 215 is generally configured to generate tasks in view of the configuration of the manipulator system, the generative nature of LLMs and LMMs may still result in the generation of tasks that cannot be performed. Accordingly, the task filtering model 222 may function as a sanity check mechanism on the task generation model 215 to ensure only feasible tasks are implemented. Additionally, a user may provide user input include user preferences and the task filtering model 222 may further evaluate the user preferences and filter the tasks according to the user preferences.

The task filtering model 215 may filter the tasks according to the risk values associated with each task. For example, the task filtering model 215 may determine that a task that would typically be performed autonomously may instead be perform semiautonomously if the risk value (e.g., the single risk value, a particular component risk value, or a composite risk value) is below a first threshold. Similarly, if the risk value is further below a second threshold value, the task filtering model 215 may instead indicate the task it to be performed manually. It should be appreciated that the task selection constitution 221 may include rules defining the thresholds and the corresponding task categorization impacts.

In a second stage, the remaining tasks are input into a task selection model 225 via a prompt that also includes the task selection constitution 221. In some embodiments, the task selection model 225 is an LLM. The task selection model 225 is configured to analyze the potential tasks or sets of tasks to be performed and associated risk values and select a preferred task or set of tasks to implement. Because the task filtering model 222 may have categorized the input tasks based on the risk value for the task, the tasks the task selection model 225 may select between may be safer than conventional techniques. Notwithstanding the foregoing, when selecting between multiple possible tasks (or sets of tasks) that can be performed, the task selection model 225 may be configured to select tasks with lower risk values. It should be appreciated that the task selection constitution 221 may define the rules by which the task selection model 225 selects tasks in view of the risk values.

The task selection model 220 may provide the selected tasks to a user along with associated risk values for the user to evaluate. The user may determine that certain risk values are acceptable, or unacceptable, and may provide user feedback indicative of one or more changes to a selected task. For example, the user may provide input indicative of a currently selected task that is to be performed manually, that the user indicates should be performed semi-autonomously, autonomously, or not at all. The user input may then be used as input to the task generation module 210 and/or the task selection module 220 to further regenerate tasks (e.g., additional tasks, different tasks, etc.,) associated risk values, and filter and select a new set of tasks given the updated generated tasks and risk values based on the user input.

In examples, the task selection model 225 may implement conformal prediction techniques to generate a certainty metric that is indicative of an amount of certainty that an autonomous, or semi-autonomous task, task will be performed, according to expectations, by a robotic system or device. The task selection model 225 may determine the certainty metric from the filtered tasks, associated risk values, and the plurality of data streams (e.g., the available types of data streams, the quality of data, etc.). The task selection module 220 may provide a task along with an associated certainty metric to a user via a user interface or display, and the user may evaluate whether a robotic system should perform the task based on the provide certainty metric. The user may provide an input indicative of the task to be performed by a robotic system (e.g., autonomously or semi-autonomously,) or the user may provide an input to the system that the task should not be performed by a robotic system. To determine whether the task should be performed by a robotic system, the user may compare the certainty metric to a threshold value. In examples, the task selection model 225 may itself compare a determined certainty metric to a threshold value and further filter tasks to be performed by robotic structures, and tasks not to be performed by robotic structures based on associated task certainty values and the certainty value threshold.

The task selection model 225 may be a foundational machine learning model that is fine-tuned to filter the tasks, and select task, based on the input generated tasks and associated risk values. Similar to the training of the task generation model 215, the task selection model 225 may be trained using a Q-learning and conformal prediction techniques. However, during the tuning process for the task selection process, the task selection model 225 may be trained based on a recall metric indicative of a percentage of tasks correctly categorized as being tasks that cannot be performed by the repositionable structures.

The task selection model 220 then outputs the selected task(s) and provides the selected task(s) to the task assignment module 230, and the robotic action module 250 accordingly.

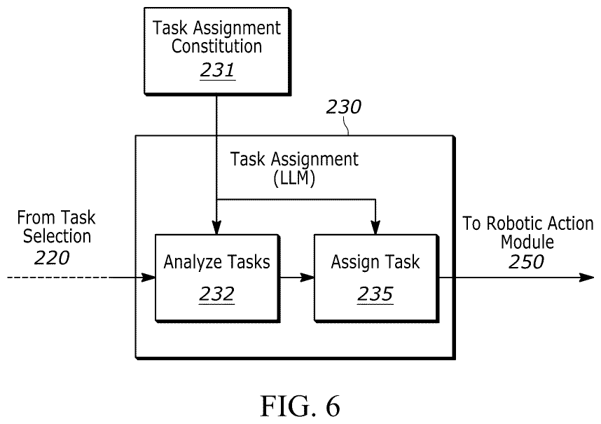

FIG. 6 is a schematic diagram of an example task assignment module 230 of FIG. 2 for assigning manual and/or semiautonomous tasks (such as the tasks selected by the task selection module 220) associated with a medical procedure. Accordingly, the task assignment module 230 may be communicative coupled to the task selection module 220. As shown in FIG. 2, the task assignment module 230 may be configured to receive the selected tasks from the task selection module 220 and the task assignment constitution 231 as an input.

As illustrated, the task assignment module 230 may include a task analysis model 232 configured to analyze the selected tasks and determine personnel to perform the selected tasks. The task analysis model 232 may be configured to determine the personnel and/or robotic systems and devices to perform various tasks according to rules of the task assignment constitution 231. In some embodiments, the task analysis model 232 is an LLM. In these embodiments, the task analysis model 232 may be configured to generate a prompt to the LLM that asks the LLM to identify the various personnel, equipment, robotic systems, etc. required to perform the input task. For example, the task analysis model 232 may identify a type of task associated with the input task (e.g., a repositioning task, an end effector activation task, a configuration task, etc.) and identify which personnel and/or skillsets or qualifications thereof are required to perform the input task. As one example, an anesthetist may be required for performing certain tasks (administering the anesthesia), while the anesthetist is not required for other tasks (manually repositioning a manipulator). The task analysis model 232 may determine various personnel, devices, systems, for performing a task based on one or more risk values associated with the various tasks to be performed. Additionally, the task assignment model 232 may determine various personnel, devices, and systems for performing tasks based on the category of each task as determined by the task filtering model 222 (e.g., autonomous, semi-autonomous, or manual).

After identifying the requirements to perform the task and/or subtasks thereof, the task assignment module 230 may input the tasks and the data streams 202 and the task assignment constitution 231 into the task assignment model 235. It should be appreciated that the task assignment model 235 may be the same or different LLM as the task analysis model 232. In embodiments where the same LLM is used for both models 232, 235, the functionality of the models 232, 235 may be achieved via a single prompt to the LLM. In these embodiments, the third constitution 231 input to the LLM with the prompt may include additional rules that govern the task assignment process.

In examples, the task assignment constitution 231 includes a set of rules defining how the LLM is to analyze the tasks to identify the required equipment and/or personnel for each task. As one example, the task assignment constitution 231 may include rules that define how to select between multiple instruments capable of performing a task. As one example, the task assignment constitution 231 may include rules the state that the instrument that has to move the least to accomplish the task should be assigned the task. As another example, the task assignment constitution 231 may include user preference rules that define how the user prefers the tasks be assigned (e.g., to a manipulator arm and/or instrument teleoperated by the user's dominant hand). As another example, tasks with risk scores below a threshold value may only be assigned to personnel with additional qualifications than would otherwise be acceptable. Accordingly, when the input task and the task assignment constitution 231 are input to the LLM, the LLM may output the task assignment that complies with the user preferences.

In some embodiments, if the task involves semi-autonomous or fully autonomous implementation, the task assignment module 270 may provide an indication of the task to the robotic action module 250 for implementation via robotic structures, instruments, and auxiliary devices at the appropriate time.

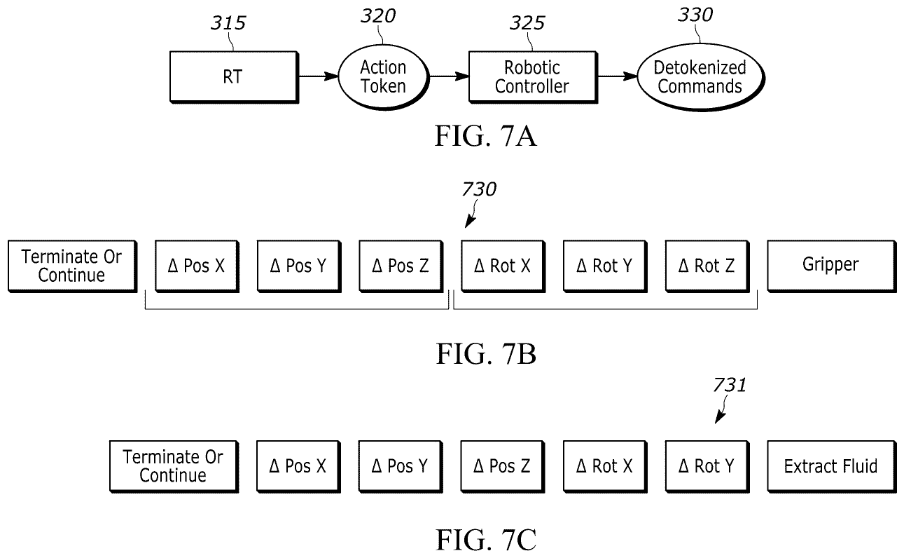

FIG. 7A is a schematic diagram illustrating detokenizing action tokens to control a repositionable structure (such as the repositionable structures 120 of FIG. 1), an instrument, or an auxiliary device to implement a semi-autonomous task. Detokenization is a process under which a system converts an action token 320 (e.g., a text string or commands) output by the robotics transformer model 315 into actual robotic commands 330 (an “action sequence”) to control the indicated equipment, for example, by changing the pose or by activating or otherwise controlling a functionality supported by the indicated equipment.

As described above, the RT model 315 may be fine-tuned and/or selected based on the particular manipulator system model. As such, knowledge of the capabilities of the manipulator system are incorporated into the RT model 315 itself. Similarly, the RT model 315 may be configured to maintain a list of current equipment coupled to the control system. Accordingly, the action tokens 320 output by the RT model 315 may include a textual indication of the equipment that is to perform the action. For example, an output action token 320 may state “Move Instrument X coupled to Arm A to target work area” or “Use Instrument Y to cauterize incision.” Accordingly, the action tokens 320 and detokenization are specific to a robotic system or model based on the capabilities of a given robotic system.

In some embodiments, the action tokens 320 may be further tailored to the current state of the manipulator system. For example, the RT model 315 may be configured to accept a kinematic data stream as an input such that the output action tokens indicate a pose for the instrument and/or arm in the robotic coordinate system. Accordingly, as one example, rather than outputting an action token 320 indicating that an instrument should move to the target work area, the output action token 320 may instead indicate “Move Instrument X to position (x, y, z) and orientation (a, B, Y).” As a result, the action tokens 320 output by the RT model 315 are tailored to the specific state of the manipulator system improving the ability of the control system to accurately interpret and implement autonomously-generated control commands.

Similarly, because the RT model 315 is aware of the functionality supported by the instruments coupled to the control system, the RT model 315 is able to output commands that are specifically tailored to the implementing instrument. For example, rather than outputting an action token that states “Clamp blood vessel,” the RT model 315 may instead output an action token 320 that states “Place gripper around blood vessel at position (x, y, z) and engage grip to exert 50 pascals of force.” As a result, the RT model 315 is able to generate action tokens 320 that have improved alignment with the actual functionality supported by the control system, thereby enabling more precision in the generation of the action tokens 320 and more robust usage of instrument functionality.

After the RT model 315 generates the action tokens 320, the action tokens 320 are input into the robotic controller 325 to convert the natural language action tokens into de-tokenized commands 330 (e.g., parameterized commands, such as PID parameter values or MPC parameter values, used by the control system actually realize the instructed actions). With simultaneous reference to FIGS. 7B and 7C, depicted are example parameterized command structures utilized by the control system.

The de-tokenized command 730 of FIG. 7B is configured to control operation of a gripper instrument that has 6 motive degrees of freedom (DOFs). Accordingly, the de-tokenized command 730 may include deltas by which the control system is to change each DOF.

Additionally, the de-tokenized command 730 includes additional DOFs related to the functionality supported by the instrument. For example, in the illustrated example related to a gripper, the “gripper” DOF may indicate an amount of force the gripper should exert. Similarly, the de-tokenized command 731 of FIG. 7C is configured to control operation of a fluid extraction instrument that has 5 motive degrees of freedom (DOFs) and one functional DOF. It should be appreciated that the length of the de-tokenized control commands 330 may vary based on the number of motive and functional DOFs supported by the controlled equipment. Accordingly, the length of the de-tokenized commands 330 have fewer unused parameters, thereby enabling more efficient usage of control buses.

In some embodiments, the de-tokenized commands 330 may also control one or more user feedback devices, such as a display device. For example, a de-tokenized command 330 may be configured to cause a display unit to provide an instruction for executing a task that is to be performed manually or semi-autonomously by a user.

While FIGS. 2-7C describe a process for performing task filtering and assignment based on risk assessment to implement one or more tasks, it should be appreciated that the disclosed process may be repeated throughout the procedure until its completion. As a result, each stage of the procedure may be discretized into tasks, that are converted into de-tokenized commands such that any portion of the procedure can be implemented via closed-loop autonomous control, semi-autonomous control, or manually by personnel. It should be appreciated that in some embodiments, the modules 210, 220, and/or 230 may analyze the multimodal data stream 202 to anticipate future tasks, and assign the future tasks, that are predicted to be performed based on a current state. Thus, the tasks analyzed by the modules 210, 220, and/or 230 may differ from the task being implemented by the robotic action module 250 or by personnel. As a result, the control system is able to anticipate future actions and future task assignments in order to ensure closed-loop control of the overall procedure occurs more efficiently.

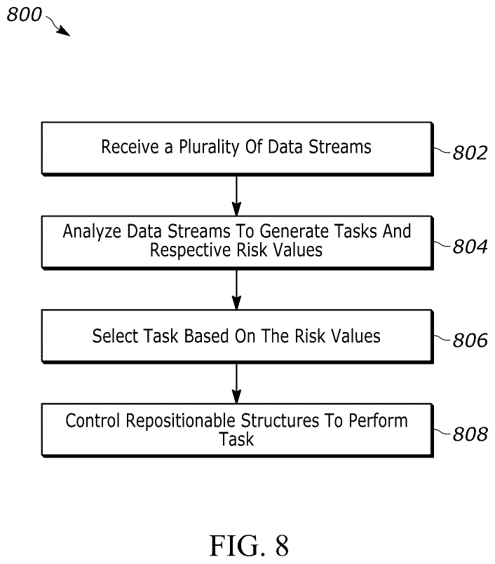

FIG. 8 is a flow diagram of a method 800 for performing risk-based task selection. The method 800 may be performed by a processor system or a control system (such as the processor system 150 and control system 140 of FIG. 1). In some embodiments, the control system may implement an AI-assist module (such as the AI-assist module 180) to perform the functionality described with respect to the machine learning models. In implementations, the method 800 may further be performed with a system including one or more repositionable structures (such as the repositionable structures 120) that may be operably coupled to one or more instruments (such as the instruments 122).

The method 800 may begin at block 802 when the control system receives a plurality of data streams (such as the multimodal data streams 202) from one or more data sources. The data streams may include multi-modal data streams of different types of data, as provided by different devices or systems. For example, the multi-modal data streams may include data from a video camera, audio data, force sensor data, system events (such as the events data stream 252d), endoscopic image data (such as the procedure video data stream 252a), operating room image data, kinematics data (such as the kinematics data stream 252b), haptics data, force data (such as the force data stream 252c), shape sensing data, tissue impedance data, environmental data, personnel procedure history, phase information, task information, personnel training data, a data stream indicative of available instruments, intraoperative image data, user inputs, and user preference data. Accordingly, the one or more data streams may include environmental data including one or more of data indicative of an interaction between the system and its environment, force data associated with instrument contact with patient tissue, force data associated with feedback to one or more manipulators, personnel present in a medical environment, and data indicative of system collisions with objects in the environment. In some embodiments, the one or more data streams may include user preference data.

At block 804, the control system may analyze, via a task generation machine learning model, one or more data streams from the plurality of data streams to generate (i) one or more tasks that may be performed by the one or more repositionable structures and (ii) respective risk values associated with the one or more tasks. In some embodiments, the risk values are the risk values are determined based on the plurality of data streams and/or a quality of the data provided by a data stream of the plurality of data streams. In some embodiments, the task generation machine learning model may determine a certainty metric associated with a generated task. In these embodiments, the risk values for the generated task may be based on the certainty metric.

To analyze the plurality of data streams, the control system may be configured to input a task generation constitution including a plurality of rules into the task generation machine learning model to control how the task generation machine learning model analyzes the data streams to generate the one or more tasks. For example, the task generation machine learning model may include foundational rules that define allowable robotic actions (e.g., limitations on instrument operation for patient and user safety, such as Asimov's three rules), safety rules that define safe operation of the computer-assisted medical system (e.g., spatial limitations, sanitization standards, etc.), embodiment rules that define capabilities and limitations of the computer-assisted medical system, associated instruments and devices (e.g., degrees of freedom of robotic arm movement, imaging capabilities of endoscopes and imaging devices, volume capabilities of extraction and injection devices, etc.), procedural rules operating to the guidelines for a given procedure (e.g., rules specific to performing urological procedures, cardiac procedures, general practice, etc.), and/or user preference rules (e.g., rules defined and input by a user or personnel for performing a procedure or task). Accordingly, the task generation machine learning model may generate the natural language outputs according to the set of rules of the task generation constitution.

In some embodiments, the task generation machine includes a vision-language model (VLM), a vision foundational model (VFM), or a large language model (LLM). Accordingly, the task generation machine learning model may be trained or fine-tuned using a conservative Q-learning or conformal prediction techniques. In these embodiments, the task generation machine learning model may be trained or fine-tuned based on a safe task metric indicative of a percentage of tasks proposed by the task generation machine learning model that are safe and feasible to be performed by the repositionable structures.

At block 806, the control system may select, via a task selection machine learning model (such as the task selection model 225 of FIG. 2), an automated task based on the respective risk values. To select the automated task, the control system may be further configured to categorize the tasks into categories including (i) automated tasks to be performed by the repositionable structures, and (ii) tasks that cannot be performed by the repositionable structures; and select the automated task based on the filtered tasks. To perform the filtering, a task selection constitution (such as the task selection constitution 221 of FIG. 2) defining one or more levels of risk and how to select the tasks based on the one or more levels of risk may be input into the task selection model along with the generated tasks. For example, the task selection constitution may include rules that (i) categorize autonomous tasks with a risk value below a first risk threshold as semiautonomous tasks, and/or (ii) categorize autonomous or semiautonomous tasks with a risk value below a second confidence threshold as tasks to be performed manually by users or surgical personnel, wherein the second risk threshold is lower than the first risk threshold.

In some embodiments, the task selection machine learning model is an LLM. Accordingly, in some embodiments, the task selection machine learning model is trained or fine-tuned based on a recall metric indicative of a percentage of tasks correctly categorized as being tasks that cannot be performed by the repositionable structures.

In some embodiments, the risk values of the selected tasks are presented to a user. For example, the user may indicate that the risk is acceptable or that the control system should select alternative tasks instead.

At block 808, the control system may be configured to control the one or more repositionable structure to perform the selected automated task. In some embodiments, the selected automated task is an semiautonomous task that is performed by the repositionable structures with user assistance. In these embodiments, the control system may provide indications to one or more users of the semiautonomous tasks; and control the repositionable structure to perform the semiautonomous tasks in coordination with the one or more users.

One or more components of the examples discussed in this disclosure, such as control system 140, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 502.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain examples and examples have been described above and shown in the accompanying drawings, it is to be understood that such examples and examples are merely illustrative and are not limited to the specific constructions and arrangements shown and described, since various other alternatives, modifications, and equivalents will be appreciated by those with ordinary skill in the art.

What is claimed is:

1. A computer-assisted system for risk-based task selection, the system comprising:
one or more repositionable structures configured to support respective instruments; and
a control system operably coupled to the repositionable structure, wherein the control system is configured to:
receive a plurality of data streams from one or more data sources;
analyze, using a task generation machine learning model, the data streams to generate (i) one or more tasks that may be performed by the one or more repositionable structures and (ii) respective risk values associated with the one or more tasks, wherein a task generation constitution including a plurality of rules is input into the task generation machine learning model to control how the task generation machine learning model analyzes the data streams to generate the one or more tasks;
select, using a task selection machine learning model, an automated task based on the respective risk values; and
control the one or more repositionable structures to perform the selected automated task.

2. The computer-assisted system of claim 1, wherein the task generation constitution includes foundational rules, safety rules, embodiment rules, or procedural rules, and wherein at least one of:
the foundational rules include rules pertaining to allowable robotic actions,
the safety rules include rules pertaining to tasks that are considered safe or unsafe based on the data streams and capabilities of the one or more repositionable structures or the respective instruments, and
the embodiment rules include rules pertaining to limitations of the repositionable structures and the instruments.

3. The computer-assisted system of claim 2, wherein the embodiment rules include rules pertaining to one or more degrees of freedom of motion of the repositionable structures and the instruments, one or more capabilities of the instruments, a field of view of an imaging instrument, one or more payloads delivered by the computer-assisted system, a capability or limitation of one or more sensors communicatively coupled to the control system.

4. The computer-assisted system of claim 1, wherein the automated tasks include semiautonomous tasks that are performed by the repositionable structures with user assistance, and wherein the control system is further configured to:
provide indications to one or more users of the semiautonomous tasks; and
control the repositionable structure to perform the semiautonomous tasks in coordination with the one or more users.

5. The computer-assisted system of claim 1, wherein the plurality of data streams includes one or more of endoscopic image data, operating room image data, kinematics data, haptics data, force data, shape sensing data, environmental data, intraoperative imaging data, personnel identification data, personnel procedure history data, and personnel training data.

6. The computer-assisted system of claim 1, wherein to select the automated task the control system is further configured to:
filter, using the task selection machine learning model, the tasks based on the associated risk values to categorize the tasks into categories including (i) automated tasks to be performed by the repositionable structures, and (ii) tasks that cannot be performed by the repositionable structures; and
select the automated task based on the filtered tasks.

7. The computer-assisted system of claim 1, wherein a data stream of the plurality of data streams includes a set of user preferences and wherein to select the automated task the control system is further configured to:
evaluate the user preferences and select the automated task based on the user preferences.

8. The computer-assisted system of claim 1, wherein the task generation machine learning model includes a vision-language model (VLM), a vision foundational model (VFM), or a large language model (LLM).

9. The computer-assisted system of claim 1, wherein the task generation machine learning model is trained or fine-tuned based on or using one or more of a conservative Q-learning or conformal prediction techniques; a safe task metric indicative of a percentage of tasks proposed by the task generation machine learning model that are safe and feasible to be performed by the repositionable structures; or a recall metric indicative of a percentage of tasks correctly categorized as being tasks that cannot be performed by the repositionable structures.

10. The computer-assisted system of claim 1, wherein the control system is further configured to:
determine, via the task generation machine learning model, a certainty metric associated with a generated task,
wherein the risk values for the generated task are based on the certainty metric.

11. The computer-assisted system of claim 1, wherein the control system is further configured to:
provide, to a user, an indication of the risk values.

12. The computer-assisted system of claim 1, wherein the risk values are based on one or more of the plurality of data streams; or a quality of the data provided by a data stream of the plurality of data streams.

13. The computer-assisted system of claim 1, wherein the control system is further configured to:
filter, via the task selection machine learning model, the tasks based on respective risk values for each task.

14. The computer-assisted system of claim 1, wherein to select the tasks based on the respective risk values, the control system is further configured to:
input, into the task selection machine learning model, a task selection constitution defining one or more levels of risk and how to filter the tasks based on the one or more levels of risk.

15. The computer-assisted system of claim 14, wherein to select the tasks, the control system is further configured to:
categorize, via the task selection machine learning model, autonomous tasks with a risk value below a first risk threshold as semiautonomous tasks.

16. The computer-assisted system of claim 15, wherein to select the tasks, the control system is further configured to:
categorize, via the task selection machine learning model, autonomous or semiautonomous tasks with a risk value below a second confidence threshold as tasks to be performed manually by users or surgical personnel, wherein the second risk threshold is lower than the first risk threshold.

17. A method for risk-based task selection via a computer-assisted robotic system comprising one or more repositionable structures configured to support respective instruments, and a control system operatively coupled to the one or more repositionable structures, the method comprising:
receiving a plurality of data streams from one or more data sources;
analyzing, using a task generation machine learning model, the data streams to identify one or more tasks that may be performed by the one or more repositionable structures and determine respective risk values associated with the one or more tasks, wherein a task generation constitution includes a plurality of rules is input into the task generation machine learning model to control how the task generation machine learning model analyzes the data streams;
selecting, using a task selection machine learning model, an automated task based on the respective risk values; and
controlling the one or more repositionable structures to perform the selected automated task.

18. The method of claim 17, wherein filtering the tasks further comprises filtering the tasks into semiautonomous tasks that are performed by the repositionable structures with user assistance, and further comprising:
providing indications to one or more users of the semiautonomous tasks, and
controlling the repositionable structure to perform the semiautonomous tasks in coordination with the one or more users.

19. The method of claim 17, wherein selecting the tasks based on the risk values comprises:
inputting, into the task selection machine learning model, a task selection constitution defining one or more levels of risk and how to filter the tasks based on the one or more levels of risk;
categorizing, via the task selection machine learning model, autonomous tasks with confidence ratings below a first risk threshold as semiautonomous tasks; and
categorizing, via the task selection machine learning model, autonomous or semiautonomous tasks with a risk value below a second confidence threshold as tasks to be performed manually by users or surgical personnel, wherein the second risk threshold is lower than the first risk threshold.

20. One or more non-transitory, computer-readable media storing instructions that, when executed by a control system of a computer-assisted system, causes the control system to:
receive a plurality of data streams from one or more data sources;
analyze, using a task generation machine learning model, the data streams to generate (i) one or more tasks that may be performed by one or more repositionable structures of the computer-assisted system and (ii) respective risk values associated with the one or more tasks, wherein a task generation constitution including a plurality of rules is input into the task generation machine learning model to control how the task generation machine learning model analyzes the data streams to generate the one or more tasks;
select, using a task selection machine learning model, an automated task based on the respective risk values; and
control the one or more repositionable structures to perform the selected automated task.
